(19) United States
(12) Patent Application Publication
(10) Pub. No.: US 2003/0233118 A1
(43) Pub. Date: Dec. 18, 2003

(54) Method for treating congestive heart failure using external counterpulsation

(76) Inventor: John C. K. Hui, East Setauket, NY (US)

Correspondence Address: HARNESS, DICKEY & PIERCE, P.L.C., P.O. BOX 828, BLOOMFIELD HILLS, MI, 48303, US

(21) Appl. No.: 10/189,788

(22) Filed: Jul. 3, 2002

Related U.S. Application Data
(60) Provisional application No. 60/388,545, filed on Jun. 13, 2002.

(51) Int. Cl. A61B 17/00

(52) U.S. Cl. 606/201

(57) ABSTRACT

A heart failure treatment method includes administering external counterpulsation therapy sessions to a subject from about 10 to about 80 days. Each counterpulsation therapy session is preferably from about 20 to about 90 minutes. Administering external counterpulsation therapy includes providing pressure devices adapted to be received about the lower extremities of the subject, interconnecting a source of compressed fluid with the pressure devices, interconnecting a fluid distribution assembly with the pressure devices and the source of compressed fluid, distributing compressed fluid from the source of compressed fluid to the pressure devices, providing a controller in communication with the fluid distribution assembly, and inflating and deflating the pressure devices using the controller so as to minimize end diastolic pressure.

### CROSS-REFERENCE TO RELATED APPLICATIONS

This application claims the benefit of U.S. Provisional Application No. 60/388,545, filed Jun. 13, 2002.

### FIELD OF THE INVENTION

The present invention relates to external counterpulsation, and more particularly, to the use of external counterpulsation for the treatment of congestive heart failure.

### BACKGROUND

Heart failure is a complex clinical syndrome manifested by abnormal heart function resulting in inadequate cardiac output for metabolic needs. Cardinal symptoms include shortness of breath (dyspnea) and fatigue. It may also be accompanied by edema and fluid in the lungs (in “congestive” heart failure). It is one of the most common causes of disability and death; three to four million individuals are diagnosed with heart failure in the United States alone. Nearly half of the patients whose symptoms become moderately severe are dead within two years. It is the proximate cause of death in several hundred thousand people every year.

Heart failure may be a primary disorder, or secondary to other circulatory problems. The most common cause of heart failure is atherosclerosis which causes blockages in the blood vessels (coronary arteries) that provide blood flow to the heart muscle. Such blockages may cause myocardial infarction, with subsequent decline in heart function. Other causes of heart failure include valvular heart disease, long-standing hypertension, cardiac arrhythmias, hyperthyroidism, viral infections of the heart, excessive alcohol consumption, and diabetes. Yet other cases of heart failure are idiopathic, without any clear etiology. On physical examination, patients with heart failure tend to have elevations in heart and respiratory rates, rales (an indication of fluid in the lungs), edema, jugular venous distension, and enlarged hearts. Heart failure is also typically accompanied by alterations in one or more aspects of beta-adrenergic function.

A variety of treatments for heart failure are known in the art. They include pharmacological therapies, coronary revascularization procedures (e.g. coronary artery bypass surgery and angioplasty), and heart transplantation. Pharmacological therapies are directed toward increasing the force of contraction of the heart (by using inotropic agents such as digitalis and beta-adrenergic receptor agonists), reducing fluid accumulation in the lungs and elsewhere (by using diuretics), and reducing the work load of the heart (by using agents that decrease systemic vascular resistance such as angiotensin converting enzyme inhibitors). While such drug treatments can improve symptoms, and potentially prolong life, the prognosis in most cases is poor. There remains to be found a modality that actually treats heart failure with long-lasting results. Thus, there remains a keen need for new treatments to treat heart failure.

External counterpulsation is a noninvasive, atraumatic means for assisting and increasing circulation in patients. Disorders treated with external counterpulsation include angina, obstructive coronary artery disease, acute myocardial infarction, and cardiogenic shock. External counterpulsation involves the inflation and deflation of sets of compressive fluid (e.g., air) cuffs wrapped around a patient's extremities (e.g., calves, lower thighs and/or upper thighs) to create a retrograde arterial pressure wave and increase venous blood return. Timing of the inflation of the fluid cuffs is modulated by physiological signals related to the patient's heart cycle (e.g., via electrocardiography). Timing of inflation and deflation is adjusted so that the flow of blood from the extremities reaches the heart at the onset of diastole. The result is augmented diastolic central aortic pressure and increased venous return. Moreover, rapid, simultaneous deflation of the cuffs produces systolic unloading. External counterpulsation treatments and devices are described in the art, including U.S. Pat. No. 3,303,841, Dennis, issued Feb. 14, 1967; U.S. Pat. No. 3,403,673, MacLeod, issued Oct. 1, 1968; U.S. Pat. No. 3,654,919, Birtwell, issued Apr. 11, 1972; U.S. Pat. No. 3,866,604, Curless et al., issued Feb. 18, 1975; U.S. Pat. No. 4,753,226, Zheng et al., issued Jun. 28, 1988; U.S. Pat. No. 5,554,103, Zheng et al., issued Sep. 10, 1996; U.S. Pat. No. 5,997,540, Zheng et al., issued Dec. 7, 1999; PCT Application WO 99/08644, Shabty et al., published Feb. 25, 1999; Zheng et al., “Sequential External Counterpulsation (SECP) in China,” Transactions of the American Society of Artificial External Organs, 29:599-603 (1983); Soroff, et al., “Historical Review of the Development of Enhanced External Counterpulsation Therapy and its Physiologic Rationale,” Cardiovascular Reviews & Reports (1997); Stroebeck et al., “The Emerging Role of Enhanced External Counterpulsation in Cardiovascular Disease Management,” Cardiovascular Reviews & Reports (1997); Chou, “Enhanced External Counterpulsation,” ACC Educational Highlights (1998); Arora, et al., “The Multicenter Study of Enhanced External Counterpulsation (MUST-EECP): Effect of EECP on Exercise-induced Myocardial Ischemia and Anginal Episodes,” J. Am. Coll. Cardiology, 33:No. 7 (1999); and Soran et al., “Enhanced External Counterpulsation in the Management of Patients with Cardiovascular Disease,” Clin. Cardiol. 22:173-178 (1999).

### SUMMARY OF THE INVENTION

The present invention provides methods for treating heart failure in a human or animal subject, comprising administering a plurality of external counterpulsation therapy sessions to the subject during a period of from about 10 to about 80 days. Preferably, the sessions comprise treatment for from about 30 to about 200 minutes per day of treatment. In one embodiment, the subject has systolic heart failure. In another embodiment, the subject has diastolic heart failure.

In a preferred method, the pressure devices used in the therapy are inflated and deflated so as to minimize end diastolic pressure, whereby an increased cardiac output in the subject is achieved. Preferably, diastolic augmentation is maximized. Also, preferably, the pressure devices are inflated and deflated so as to maximize systolic unloading. Also, preferably, the subject's blood oxygen level is measured during treatment.

It has been found that the methods of this invention afford benefits including long-term improvement in cardiac function as well as improvement in the symptoms of heart failure, while avoiding significant side effects. Specific benefits and embodiments of the present invention are apparent from the detailed description set forth herein. It should be understood, however, that the detailed description and specific examples, while indicating embodiments among those preferred, are intended for purposes of illustration only and are not intended to limit the scope of the invention.

### BRIEF DESCRIPTION OF THE DRAWINGS

Exemplary embodiments of external counterpulsation apparatus useful in the methods of this invention are depicted in FIGS. 1-12.

In particular, FIG. 1 is a diagrammatic view of an external counterpulsation apparatus.

FIG. 2 is a diagrammatic representation of the fluid (air) handling system of an external counterpulsation apparatus.

FIG. 3 is a diagram for a control mechanism for an external counterpulsation apparatus.

FIG. 4 is a graphic representation of the relationship between a subject's electrocardiogram, the sequential valve opening signals and the pressure device inflation pressure waveforms during operation of an external counterpulsation apparatus.

FIG. 5 diagrammatically illustrates initiation timing logic for inflation/deflation.

FIG. 6 is a graphic representation of the interrelationships among exemplary electrocardiogram, inflation/deflation valve timing, and cuff pressure waveforms.

FIG. 7 is a graphic representation of the sequential inflation of exemplary pressure devices and the resulting blood pressure waveform.

FIG. 8 is a graphic representation of the effect of counterpulsation on blood pressure and left ventricular stroke volume.

FIG. 9 is a graphic representation of deflation time optimization.

FIG. 10 is a graphic representation of inflation time optimization.

FIG. 11 is a graphic representation of optimizing inflation time by approximation when a dicrotic notch is not detected.

FIG. 12 is a graphic representation of an exemplary external pressure waveform.

It should be noted that the drawings of devices and counterpulsation pressure waveforms set forth herein are intended to exemplify the general characteristics of external counterpulsation embodiments among those useful in the methods of this invention, for the purpose of describing such embodiments herein. These drawings may not precisely reflect the characteristics of any given embodiment, and are not necessarily intended to define or limit specific embodiments within the scope of this invention.

### DETAILED DESCRIPTION

The present invention provides methods for the treatment of heart failure in humans or other animal subjects. Such methods comprise the use of an external counterpulsation device, and may optionally use other devices and pharmaceutical treatments. Such devices and treatments useful herein must, accordingly, be therapeutically acceptable. As referred to herein, a “therapeutically acceptable” component is one that is suitable for use with humans and/or animals without undue adverse side effects (such as toxicity, irritation, and allergic response) commensurate with a reasonable benefit/risk ratio.

External Counterpulsation Method:

The methods of the present invention comprise administering external counterpulsation to a human or other animal subject having heart failure. As referred to herein, “treatment” means effecting a long-term physiological improvement in cardiac function, as well as symptomatic improvement, in a subject with a clinical diagnosis of heart failure. As referred to herein, “heart failure” is a clinical syndrome characterized by abnormal heart function with inadequate cardiac output for metabolic needs, and symptoms of shortness of breath (dyspnea), and fatigue. In one embodiment, the heart failure syndrome is systolic, associated with reduction of systolic performance of the heart and failure of the heart to pump sufficient oxygenated blood to meet the body's metabolic needs. Systolic heart failure may be characterized by enlargement or atrophy of the left ventricle, with clinical symptoms including one or more of the following: excessive sympathetic nervous system activity; tachycardia; “galloping” heart rhythm; peripheral edema; accumulation of fluid in the abdomen (ascites); and diminished urine secretion relative to intake (oliguria). In another embodiment, the heart failure is diastolic, associated with reduction of diastolic performance of the heart. Diastolic heart failure is the inability of the heart to be filled with sufficient oxygenated blood to meet the body's metabolic needs. Diastolic heart failure may be characterized by normal left ventricle systolic function, but with left ventricle hypertrophy and impaired diastolic filling. Clinical symptoms include pulmonary vascular congestion. (As used herein, the word “include” and its variants are intended to be non-limiting, such that recitation of items in a list is not to the exclusion of other like items that may also be useful in the materials, compositions, devices, and methods of this invention.) Aspects of heart failure are described in Francis, G., “Pathophysiology of the Heart Failure Clinical Syndrome,” Textbook of Cardiovascular Medicine, Chapter 79 (1998), incorporated by reference herein.

Administering external counterpulsation (herein “ECP”) to a subject comprises a method of applying external pressure to an extremity of the subject so as to create retrograde arterial blood flow and enhanced venous return from the extremity to the heart of the subject during diastole (i.e., the period of relaxation of the left ventricle of the heart). Preferably, the extremity comprises one or more of the legs of the subject, in a human subject preferably including both legs and both arms. In another embodiment, extremity in a human subject comprises both legs, more preferably including the calves, thighs, and upper thighs, and buttocks of the subject. In a preferred embodiment, the external pressure is applied using a plurality of pressure devices applied to the extremities of the subject, and inflated and deflated in synchrony with the cardiac cycle of the subject so as to create a pulse of arterial blood that arrives at the heart essentially at the end of the ejection phase of the left ventricle and closure of the aortic valve. In a preferred embodiment, the administration of ECP is performed using an external counterpulsation apparatus, preferably as described herein. (As used herein, the words “preferred” and “preferably” refer to embodiments of the invention that afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the invention.)

Preferably, the ECP therapy comprises ECP administration during a treatment period of from about 10 days to about 80 days, more preferably for from about 20 days to about 60 days. Preferably, ECP is administered on at least about 50% of the days of the treatment period (e.g., on at least about 40 days of an 80-day treatment period), more preferably on at least about 70%, more preferably at least about 85%, of the days of the treatment period. Preferably, ECP is administered at least 4 days during every 7-day period during the treatment period, such that there are no more than 3 consecutive days in which ECP is not administered. More preferably, ECP is administered at least 5 days, even more preferably at least 6 days, during every 7-day period of the treatment period. Preferably, ECP is administered for from about 30 minutes to about 200 minutes for each day during which treatment is administered, preferably from about 60 minutes to about 80 minutes per day of treatment. Preferably, the daily administration of ECP is performed in one or more sessions, for from about 20 to about 90 minutes, preferably for from about 45 minutes to about 60 minutes, more preferably for about 60 minutes per session. As referred to herein, a “session” of ECP comprises the repeated inflation and deflation of pressure devices in synchrony with the cardiac cycle of the subject in a substantially continuous manner. Preferably from 1 to 3, more preferably 1, session is conducted during each day in which ECP therapy is administered. A preferred method comprises from 1 to 3 sessions of external counterpulsation therapy during each day of at least 4 days of every 7-day period during a treatment period of from about 20 to about 60 days. Another preferred method comprises the steps of:

(a) administering to said subject an external counterpulsation therapy session lasting from about 20 minutes to about 90 minutes and repeating said therapy session for from 1 to 3 times per day for at least about 70% of the days during a treatment period of from about 20 to about 80 days; and

(b) monitoring said subject to assess the safety and or efficacy of said therapy session.

Preferably, the entire course of therapy comprises a total of from about 20 hours to about 80 hours, more preferably from about 30 hours to about 80 hours, of ECP.

In a preferred embodiment, the methods of the present invention additionally comprise the step of diagnosing the subject to confirm heart failure. Such a diagnosing step comprises performing one or more diagnostics the results of which, individually or collectively, support a clinical diagnosis of heart failure according to sound medical practice. In a method comprising such a step, the step of administering ECP is performed if, preferably only if, the diagnosis of heart failure is positive. Also, preferably, if the diagnosis is positive, the method comprises an additional step of diagnostic monitoring the subject after administering ECP, comprising performance of a diagnostic for heart failure. In a preferred embodiment, the diagnostic monitoring step comprises the same diagnostic as the diagnosing step. Preferably, the diagnostic monitoring step is performed after the administering step has been conducted for a total of at least about 20 hours. A preferred method comprises the steps of:

(a) performing a diagnostic on said subject to confirm the existence of heart failure; and, if said heart failure is confirmed;

(b) administering to said subject a plurality of external counterpulsation therapy sessions during a treatment period of from about 20 to about 60 days.

Diagnostics useful in the diagnosing steps and diagnostic monitoring steps include those selected from the group consisting of: clinical evaluation of symptoms such as dyspnea, orthonea, paroxysmal nocturnal dyspnea, chronic fatigue, peripheral edema, ascites, tachycardia, “galloping” heart rhythm, rales, jugular venous distention, and oliguria; chest radiography, computerized tomography, or coronary magnetic resonance imaging to assess the size of the heart; radionuclide ventriculogram; coronary angiography; electrocardiography, such as 12-lead ECG, signal averaged electrocardiography, and use of a Holter monitor; blood pressure monitoring; blood testing, such as complete blood count, platelet count, clotting studies, and measurement of electrolytes, BUN, creatinine, glucose, albumin, cardiac and liver enzymes, thyroid-stimulating hormone, and brain natriuretic peptide (BNP); pulse oximetry; measurement of arterial blood gases and lactate concentration; Doppler two-dimensional echocardiography (transthoracic or transesophageal); hemodynamic evaluation, (e.g., with pulmonary artery balloon catheter) for pulmonary edema; noninvasive testing for ischemia such as exercise stress testing and radionuclide stress perfusion testing; endomyocardial biopsy; and tabulation of fluid volume intake and urine output.

External Counterpulsation Apparatus:

Preferably administration of ECP is performed using an external counterpulsation apparatus (herein, “ECP apparatus”), comprising (a) one or more pressure devices that are applied to an extremity of the subject; (b) a device for inflating and deflating the pressure devices; and (c) a controller that initiates inflation and deflation of the pressure devices in synchrony with the cardiac cycle of the subject. An exemplary ECP apparatus (10) is depicted in FIG. 1, comprising three basic component assemblies, namely: a treatment table assembly (11); a pressure device (12); and control console assembly (13), preferably comprising a device for inflating and deflating the pressure devices and a controller that initiates inflation and deflation of the pressure devices. Alternative embodiments comprise one or two component assemblies.

The ECP apparatus preferably comprises pressure devices that are applied to the legs or other limbs of the subject, preferably to the calves, thighs and buttocks of the subject. Such pressure devices apply pressure to the limb using, in a preferred embodiment, a bladder that is inflated with a fluid, preferably air. Preferably the pressure device comprises a bladder and a fastener that holds the bladder against the limb, so that when the bladder is inflated pressure is applied to the limb. In a preferred embodiment, the fastener comprises a cuff body that holds the bladder against the limb, preferably a cuff surrounding a bladder. Preferably, each bladder applies from about 140 to about 320 mm Hg of pressure to the limb. The fastener is made, for example, from materials including vinyl, leather, cloth, canvas, and rigid or semi-rigid materials such as plastic or metal. Different sizes of bladders and fasteners may be provided to meet the requirements of different body shapes. Preferably space between the fastener and the bladder and between the bladder and the limb is minimized. A preferred pressure device comprises a rectangular bladder. Also, preferably, the upper and lower thigh pressure devices are a one-piece design to prevent the lower thigh pressure device from sliding during treatment.

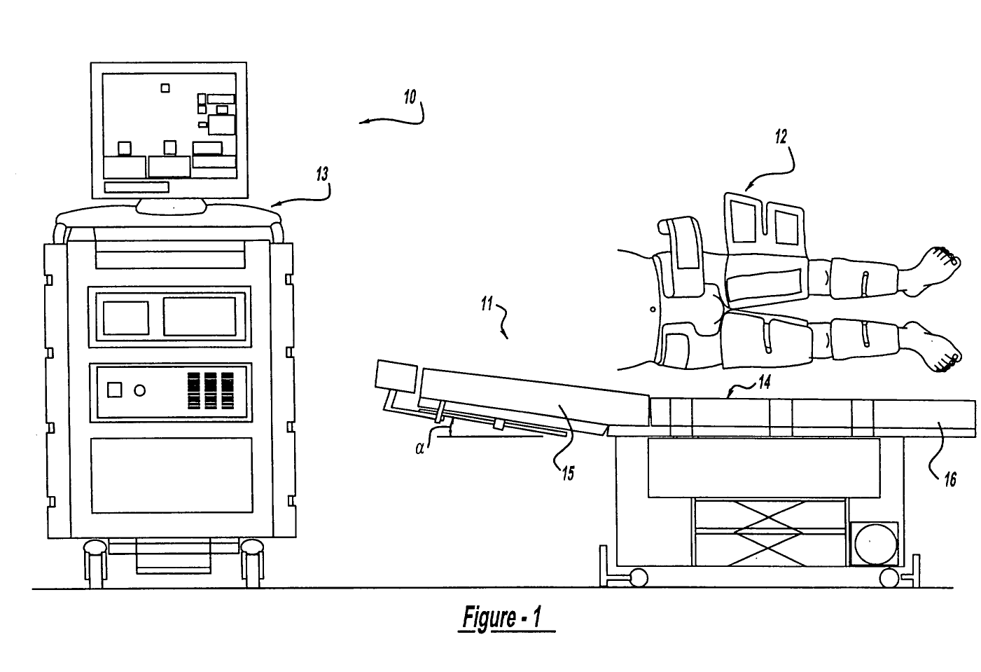

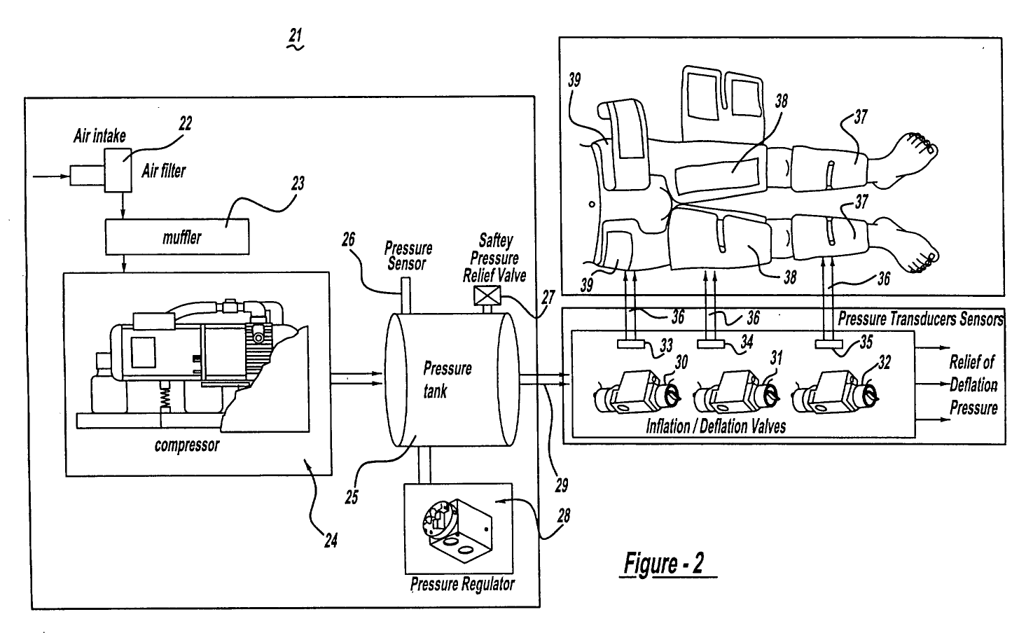

The ECP apparatus also preferably comprises a device for inflating and deflating the pressure devices using a fluid, such as air. In a preferred embodiment, where the pressure devices are inflated with air, the inflating and deflating device comprises a compressor and an air distribution mechanism that operates to distribute the air from the compressor to the pressure devices. FIG. 2 depicts a preferred embodiment of the compressed fluid (preferably compressed air) flow arrangement for the ECP apparatus (21). The apparatus generally includes an air intake/filter assembly (22), one or more mufflers (23), which can be located before or after a compressor assembly (24), a pressure tank (25), a pressure sensor/transducer assembly (26), a pressure safety relief valve (27), and a pressure regulator (28). A temperature sensor may also be included (not shown). In one embodiment, the components are housed within a cabinet or housing of the control console assembly (13). Alternatively, these components may be housed separately, such as in another housing or incorporated into the treatment table assembly (11).

A hose connection assembly (29) is used for quick connecting and disconnecting the above-described components with those mounted on, or otherwise associated with, an assembly comprising valves that individually control inflation and deflation of the pressure devices. In a preferred embodiment, the valve assembly is part of a treatment table assembly (11). Such a treatment table assembly components include a valve manifold, a number of sequentially operable inflation/deflation valves (30), (31) and (32), each with an associated pressure transducer/sensor (33), (34), and (35), respectively. A connect/disconnect assembly (36) is provided for quick and easy connection and disconnection of the inflation/deflation valves with associated pressure devices, e.g., the calf pressure devices (37), lower thigh pressure devices (38), and upper thigh pressure devices (39), respectively. In this embodiment, the inflation/deflation valves (30), (31), and (32) are a rotary actuable butterfly-type valve, which can be actuated pneumatically or electrically. In another embodiment, the valve assembly is part of the central console, and the patient may lie on any suitable table or bed.

In one embodiment, the subject may lie on an ordinary bed for treatment. In another preferred embodiment, the ECP apparatus comprises a bed as part of the treatment table assembly. As depicted in FIG. 1, the treatment table assembly (11) preferably comprises a support surface (14) having an articulating portion (15) and a horizontal portion (16). The articulating portion (15) of support surface (14) is hingedly or otherwise pivotally interconnected to the horizontal portion (16) for adjustment, either manually or by way of a power drive, to a plurality of angulated positions relative to the horizontal portion (16). The angulated position of the articulating portion (15) relative to the main horizontal portion is preferably limited to an angle a that is from about 15° to about 30° above the horizontal. Preferably, the elevation assembly comprises a motor to raise and lower the bed. Also, preferably, the treatment table assembly is configured for mobility (e.g., having wheels) from one location to another.

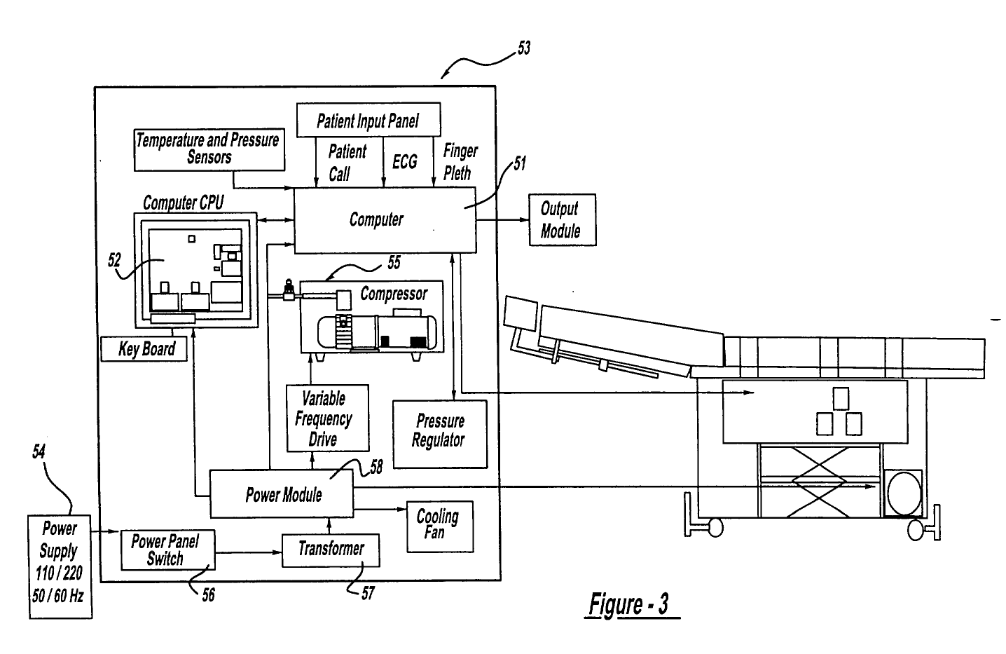

The ECP apparatus also preferably comprises a controller that initiates inflation and deflation of the pressure devices in synchrony with the cardiac cycle of the subject. In a preferred embodiment, the controller is part of a control console assembly. As depicted in FIG. 3, one control console assembly embodiment generally includes a computer (51), a user interface device (52), such as a computer monitor or touch screen, and a cabinet or housing (53), in which various system components are located and housed. The control console assembly preferably includes a power supply (54) that feeds power to the computer (51) and the compressor assembly (55) by way of a power switch panel (56), transformer (57), and power module (58), that includes a power converter and ramp-up assembly. Preferably, the control console assembly (53) is mounted on wheels for mobility from one location to another.

The user interface (52) is preferably a touch screen monitor for easy monitoring of patient treatment status, treatment parameters, and other relevant data, and provides the capability for adjustment to control operation. In one embodiment, the computer (51) monitors and records information associated with the treatment of the patient. The user interface also provides switches or computer links to switches for adjusting the timing of the inflation/deflation cycle, allowing the operator to adjust the setting of the time for the start of sequential inflation as it is measured relative to the R peak of the treated subject's ECG signal, as further described below. The apparatus may be configured so as to be self contained, i.e., with all components proximately located. Alternatively, one or more components may not be proximate, but connected to the other components through electrical or other appropriate mechanical connections. In some embodiments, the apparatus may have duplicate components (e.g., the user interface), with one component proximate to the device, and another at a remote location. In one embodiment, a user interface may be located remotely from the remainder of the device, such as in another room in the treatment facility. The computer may be located remotely from the remainder of the equipment, such as in the same facility (e.g., through remote cabelling or as part of a local area network), or in another facility (e.g., connected through an appropriate telecommunications device). The apparatus may comprise a computer proximate to the rest of the apparatus, while also being connected to another computer for remote acquisition, storage, or maintenance of data. A remote user interface may be used to control two or more devices.

In a preferred embodiment, the user interface (52) displays treatment information, including an electrocardiograph (ECG) signal from the subject being treated. As will be apparent to one skilled in the art, the R wave portion of the ECG signal is typically used to monitor the cardiac cycle of the patient. Preferably, the blood flow and/or blood pressure of the patient is also displayed, e.g., to facilitate monitoring of the cardiac cycle of the subject as well as the effect of the counterpulsation waves being applied to the ECP apparatus. In one embodiment, the signal is a photo-plethysmograph waveform signal as received from a finger plethysmograph probe.

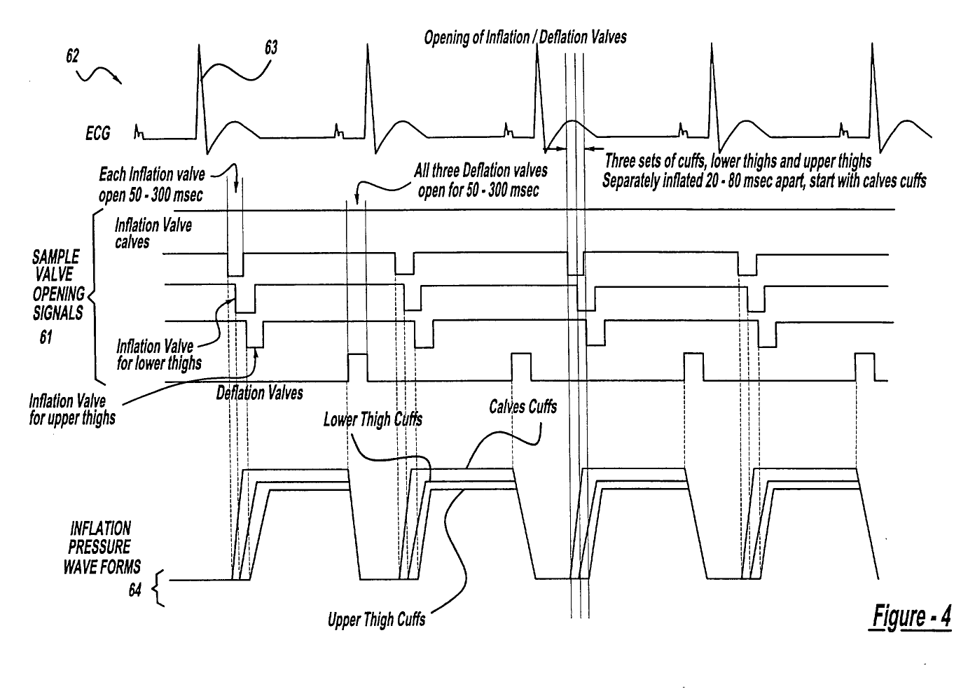

The controller initiates inflation and deflation of the pressure devices in synchrony with the subject's cardiac cycle. Inflation and deflation is effected so as to create a retrograde pulse of arterial blood that arrives at the heart at approximately the end of the ejection phase of the left ventricle at the time of aortic valve closure. The time of aortic valve closure may be determined through direct or indirect measures. In a preferred embodiment, the time of aortic valve closure is determined indirectly using finger plethysmography. The synchronization of inflation and deflation of the pressure devices is exemplified in FIG. 4. As shown, the controller generates signals (61) for opening and closing valves that release air from the pressure tank to the pressure devices. These signals are synchronized with the treated subject's ECG (62), with the R-wave (63) as a trigger point. The pressure devices are inflated sequentially as shown by pressure waveforms (64).

The safety and effectiveness of the external counterpulsation therapy depends on the precise timing of the inflation/deflation cycle in relation to the cardiac cycle of the patient. For example, a hardened arterial wall (i.e., with significant calcium deposits) will transmit the external pressure pulse up the aorta faster than an elastic artery. Therefore, the inflation valves should be opened later for a calcified artery than for a normally elastic artery. Accordingly, determination of inflation and deflation of the pressure devices is preferably adjusted for every individual subject to be treated.

In a preferred embodiment, there are several factors that are taken into account to determine the appropriate deflation time of the pressure devices. They include release of all external pressure before the next systole to produce maximal systolic unloading (maximum reduction of systolic pressure), and maintenance of inflation as long as possible to fully utilize the whole period of diastole so as to produce the longest possible diastolic augmentation (maximum increase of diastolic pressure due to externally applied pressure). Therefore, one measurement of effective counterpulsation is the ability to minimize systolic pressure, and at the same time maximize the ratio of the area under the diastolic waveform to that of the area under the systolic waveform. Also, preferably, inflation and deflation timing is adjusted to minimize end diastolic pressure. Preferably, timing is adjusted so as to maximize systolic unloading and diastolic augmentation. A preferred method comprises the treatment of diastolic heart failure by minimizing end diastolic pressure and maximizing systolic unloading and diastolic augmentation. Another preferred method comprises the treatment of systolic heart failure by maximizing systolic unloading and diastolic augmentation.

Further, there are two basic safety criteria: (1) the inflation valves are not be opened so that the pressure pulse wave reaches the root of the aorta during systole, forcing the aortic valve to close prematurely, thereby creating systolic loading; and (2) the deflation valves are opened to the atmosphere before the next R wave to allow enough time for the air pressure in the pressure devices to decay so there is no significant residual pressure causing a tourniquet effect. Preferably, the air pressure decays to a level of from about 0 to about 50 mm Hg, more preferably from about 0 to about 20 mm Hg. Finally, the inflation/deflation valves are preferably not operational when the heart rate is higher than 120 beats per minute or lower than 30 beats per minute.

In a preferred embodiment, the inflation/deflation timing control logic of the ECP apparatus is divided into two main parts: (1) the initiation stage upon power up of the apparatus, during which the inflation/deflation times are set up automatically; and (2) the operation stage during which the inflation and deflation time can be adjusted manually. In a preferred embodiment, the timing is controlled by a microprocessor.

In a preferred embodiment (as depicted in FIG. 2), there are three inflation/deflation valves (30), (31), and (32). Alternatively, there may be a separate inflation valve and a separate deflation valve for each pressure device. One inflation/deflation valve is for the calf pressure devices, one for the lower thigh pressure devices, and one for the upper thigh pressure devices. The inflation valves are normally closed, and selectively open to allow inflation when energized. Upon receipt of a signal from the inflation/deflation timing control, electrical power to the inflation valves will be switched on for a period of from about 70 to about 150 milliseconds, preferably from about 100 to about 120 milliseconds and will open them to the fluid reservoir (25). Upon receipt of the deflation valve signal, power to the deflation valves will be switched off for a period of from about 100 to about 300 milliseconds, preferably from about 100 to 150 milliseconds, and will open the valves to the atmosphere, deflating the pressure devices. The deflation valves are preferably normally open, and closed when energized, so that the pressure devices automatically deflate upon loss of power.

During the initiation stage when power is turned on, the computer will start a series of initiation procedures. An exemplary flow chart for these procedures is shown in FIG. 5. The deflation valves remain open to atmosphere. Each deflation valve will remain open for from about 100 to about 300 milliseconds, preferably for about 120 milliseconds, or long enough to relieve substantially all the air pressure from the leg and thigh pressure devices. The computer will then look for the input of the ECG and determine the presence of the QRS complex. If the QRS complex is not detected, the inflation/deflation valves will not be activated and the external counterpulsation will not start. The inflation valves will remain closed, and no compressed fluid will enter the pressure devices from the tank.

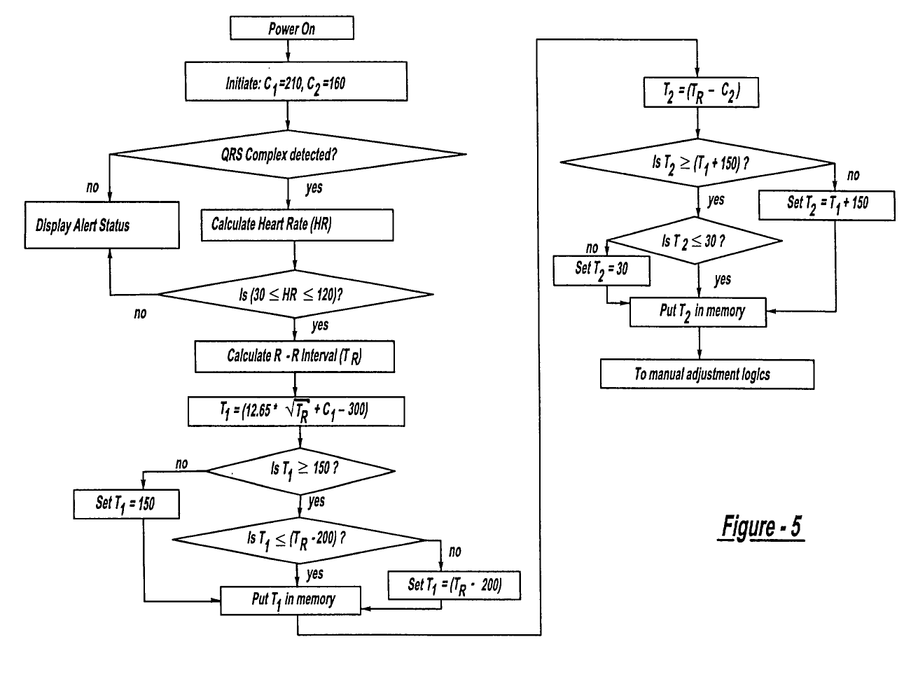

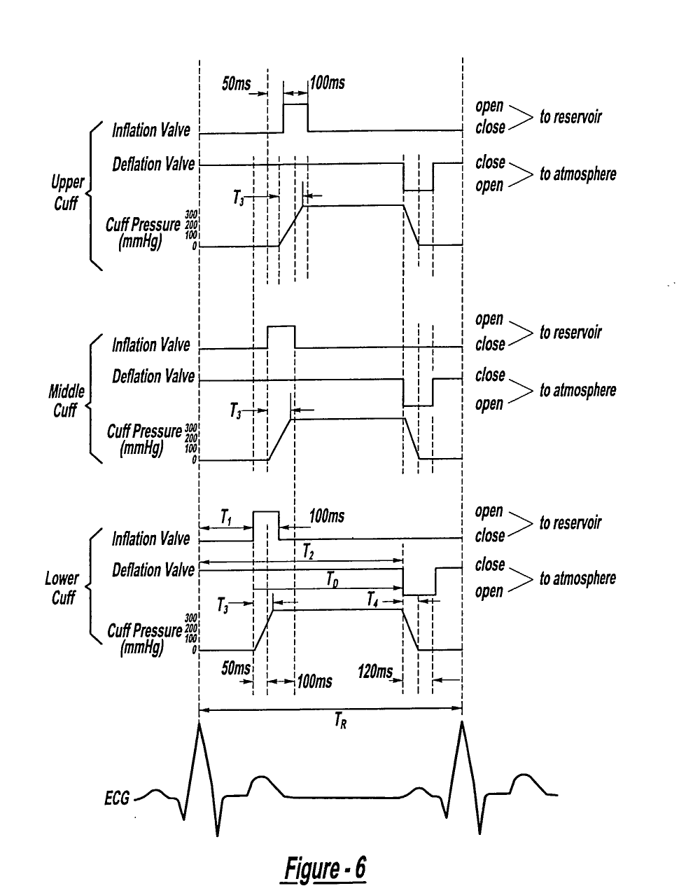

The inflation/deflation valve timing logic controls the timing of external pressure applied to the lower legs and thighs of the patient. A diagram of the timing relationship between inflation/deflation valves and ECG R wave is shown in FIG. 6, for an embodiment having the three pairs of pressure devices. The key variables for operation of the inflation and deflation valves are the inflation time (T₁) and deflation time (T₂). Definitions of T₁ and T₂ and other variables shown diagrammatically in the example of FIG. 6 are as follows.

T_{R} (R-R interval): average R-R interval in milliseconds

T₁ (inflation time): interval from R wave to the opening of lower leg inflation valve in milliseconds—Note that the inflation valve for the lower thigh pressure devices preferably opens from about 20 to about 80 milliseconds, more preferably about 50 milliseconds, after T₁. The inflation valve for the upper thigh pressure devices is preferably open for another 20 to 80, preferably 50, milliseconds after the opening of the valve for lower thigh pressure devices. In addition, inflation valves are preferably normally closed. Preferably, they will be opened for a duration of 100 milliseconds or more when energized.

T_{D} (duration time): interval between the opening of the lower leg inflation valve and the opening of the deflation valves for the pressure devices, in milliseconds

T₂ (deflation time): interval from R wave to the opening of the deflation valves, in milliseconds—Preferably, the deflation valves for the inflatable devices are normally open to the atmosphere, but are selectively closed when energized. This opening time is preferably at least about 40 milliseconds longer than the pressure decay time T₄.

T₃ (pressure rise time): interval between the time when the air pressure in the lower leg or thigh pressure devices is at its minimum (e.g., from about 0 to about 50 mm Hg) and the time when it reaches its peak pressure—This value is preferably from about 50 to about 100 milliseconds.

T₄ (pressure decay time): interval for the air pressure in the pressure devices to drop to its minimum pressure (e.g., from about 0 to about 50 mm Hg) when the deflation valves are opened to the atmosphere—The value of T₄ preferably has an average value of from about 60 to about 120 milliseconds.

In calculating the intervals for inflation and deflation, the mechanical properties of the apparatus and the physiologic properties of the subject must be considered. In this regard, in one embodiment, it takes about 20 milliseconds for the valves to fully open, about another 30 milliseconds for the air pressure to arrive at the pressure devices, about another 70 milliseconds to reach full inflation pressure, and about an additional 150 to 300 milliseconds for the applied pressure wave to travel from the vasculature of the legs and thighs to the root of the aorta. If inflation was to actually start at the closure of the aortic valve, then these time delays would result in the pulse generated by the external pressure arriving at the root of the aorta long after the end of the systolic period. For example, for a heart rate of 60 beats per minute, the systolic time is approximately 400 to 500 milliseconds per heartbeat. Therefore, for the applied pulse wave to arrive at the root of the aorta at the time the aortic valve closes, the inflation signal must start at least about 150 to 200 milliseconds after the R wave. As shown in FIG. 4, the inflation time for the lower leg pressure device starts at approximately the peak of the T wave, not at the end of the T wave. The same timing considerations apply to the opening of the deflation valves. The opening of the deflation valve occurs about 30 to 160 milliseconds before the next R wave. Since ejection of blood from the heart does not begin until about 80 milliseconds to 100 milliseconds after the R wave, and the ejected blood does not reach the extremity for another 130 milliseconds to 300 milliseconds, there is ample time for the pressure to be released from the pressure devices and for the deflation valves to close before the next systolic augmentation cycle. Therefore, because the decay time T₄ is (at most) about 120 milliseconds for the pressure devices device pressure to drop to its minimum value (e.g.,0 to 50 mm Hg), there is little or no residual pressure at the beginning of the next systolic phase, giving the peripheral vascular bed ample time to refill during cardiac systole.

In initiating therapy, the computer will determine the average interval (T_{R}), after the detection of from 3 to 8 complete R-R intervals. The computer will update T_{R} by taking the mean of the last T_{R} and the new R-R interval. Values for the inflation time (T₁) and deflation time (T₂) are then determined as follows. The initial value assigned to T₁ is based on the following formula derived from that of Bazett, Heart 7:353 (1920), which approximates the normal Q-T interval of the ECG as the product of a constant (0.4) times the square root of the R-R interval measured in seconds.

T₁=(12.65*\sqrt{T_{R}}+C₁−300) ms

In this formula, the constant 12.65 is used instead of 0.4 when converting the unit of T_{R} from seconds to milliseconds, and C₁ is a constant that is initially assigned a value equal to 210 milliseconds and may be adjusted as discussed below. The factor 300 milliseconds is equal to the approximate maximum time it takes for the applied external pressure wave to travel from the lower leg to the aortic valves.

After T₁ has been determined, it is compared to a value of 150 milliseconds. If T₁ is less than 150 milliseconds, it is then set to 150 milliseconds. If T₁ is larger than 150 milliseconds, then the calculated value will be used. This procedure guarantees that the inflation valves will not open in less than 150 milliseconds after the R wave. Even when T₁ is set at 150 milliseconds, the leading edge of the pressure wave will not arrive at the aortic root until approximately 350 milliseconds after the R wave, taking into account the time required for the pulse to travel up from the peripheral vasculature to the root of the aorta.

Once the value of T₁ has finally been determined, it is used to calculate T₂ using the following formula:

T₂=(T_{R}−C₂)

where the constant C₂ is initially set at 160 milliseconds. However, C₂ can be increased or decreased manually to achieve an optimal hemodynamic effect.

During the operation stage following the initiation stage, the values of T₁ and T₂ will be stored in memory in the controller, and used to control the inflation/deflation timing. However, the memory will be updated with every new heartbeat using the updated T_{R} to calculate the new T₁ and T₂ and stored in memory replacing the old T₁ and T_{2.} In addition, the controller will interrogate periodically (e.g., every 10 milliseconds) a flag in a register to determine if any manual adjustment has been made.

In the inflation phase of external counterpulsation, valve opening is controlled such that (i) the inflation valves furthest from the heart (distal) are opened at a time such that the pulse generated by the application of the external pressure in compressing the vascular bed travels up the arterial tree and reaches the root of the aorta when the aortic valve closes; and (ii) the second inflation valves are opened after the first valves according to a delay corresponding to when the peak of the pulse from the first pressure devices reaches the mid-point of the pressure devices controlled by the second valves (and similarly timed for the third and all other proximal pressure devices). Such a preferred embodiment for opening of the inflation valves in sequence is shown in FIG. 7. Sequential application of external pressure from the distal pressure devices to the proximal pressure devices “milks” the peripheral blood toward the heart. The sequential compression also eliminates the possibility of creating a tourniquet or “bottle neck” effect in the proximal segment of the vasculature, i.e., the occlusion of proximal arteries before distal arteries are compressed. Arrow A in FIG. 7 represents the retrograde blood flow created by the compression of pressure devices on arteries and micro-vessels. Note that the distal inflation valves open before the closure of the aorta valve as indicated by time period T because it takes approximately 100 to 300 milliseconds for the generated pulse to travel up the arterial tree.

In one embodiment, the delay in inflation between sets of pressure devices is approximated, using a fixed time interval (delay) between each set of pressure devices, e.g., ranging from approximately 20 milliseconds to 80 milliseconds, preferably about 50 milliseconds. The delay in each of the successive sets of pressure devices is not fixed in another embodiment. In such an embodiment, timing is varied because the velocity of the pulse generated by the inflation of the distal pressure devices traveling up the peripheral vascular tree to the aorta and the heart (retrograde flow) changes according to the elasticity of the arterial walls of the patients and the rate of application of the external pressure. The delay required for optimized inflation of a distal pressure devices ahead of closure of the aortic valve and delay in opening subsequent inflation valves depends on the distance of the distal pressure devices to the root of the aorta and elasticity of the aortic wall. In this manner, velocity of the generated pulse traveling up the aorta is controlled. Accordingly, distal inflation valves open at a time such that the generated retrograde pressure or flow pulse will reach the root of the aorta when the aorta closes. The next set of inflation valves opens when the pulse generated by the distal compression reaches the midpoint of its respective pressure devices as determined by a pressure detector. Any subsequent inflation valves open in a similar manner, i.e., when generated distal pulses reach the midpoint of any such pressure devices.

In one embodiment, the time delay for sequential inflation of the pressure devices is determined by first calculating the velocity of the retrograde pulse from the calf inflation device to the blood pressure detector (e.g. finger plethysmograph). This is calculated as the distance between the calf device and the pressure detector, divided by the time between inflation of the calf device and the detection of the retrograde pulse by the detector. The delay time between inflation of the calf device and the lower thigh device can then be calculated by dividing the distance between the devices by the velocity. The delay between inflation of the upper thigh inflation device and the lower thigh inflation device can be similarly calculated.

In a preferred embodiment, the ECP apparatus comprises manual inflation and deflation timing adjustment inputs, such as touch screen buttons or manual switches, as described above. Each depression of the inflation advance input triggers the controller to compare the value (T_{R}−T₁) to 200 milliseconds. If (T_{R}−T₁) is larger than 200 milliseconds, then T₁ will be lengthened by 10 milliseconds. This is done by adding 10 milliseconds to C₁ which has been initially set at approximately 210 milliseconds. The same logic is applied to limit the ability of advancing T₁ to approximately 200 milliseconds or less before the next R wave, in order to prevent the inflation valve of the calf pressure devices from opening so late that not enough time remains for the deflation valves to open before the next R wave; keeping in mind the facts that the inflation valve for the lower thigh pressure devices opens approximately 50 milliseconds after T₁ and remains open for approximately another 100 milliseconds, followed by the opening of the upper thigh pressure devices 50 milliseconds thereafter, leaving little, if any, time for the pair of deflation valves to open before the next R wave. Because the logic used in controlling the manual adjustment of the deflation valves sets a limit for the deflation to open no later than approximately 30 milliseconds before the next R wave, in the worst case scenario, the deflation valves will open to the atmosphere within approximately 30 milliseconds after the inflation valve of the upper thigh pressure devices is closed.

The other manual inflation/deflation adjustment inputs work on the same principle; that is, with each depression of one of the manual inputs, the controller will check the conditions limiting the timing of the valves, and if the limits are not reached, then the timing for the inflation/deflation valves can be advanced or retreated by subtracting or adding 10 milliseconds to C₁ or C₂ of the above equation and the equation T₂=(T_{R}−C₂) milliseconds.

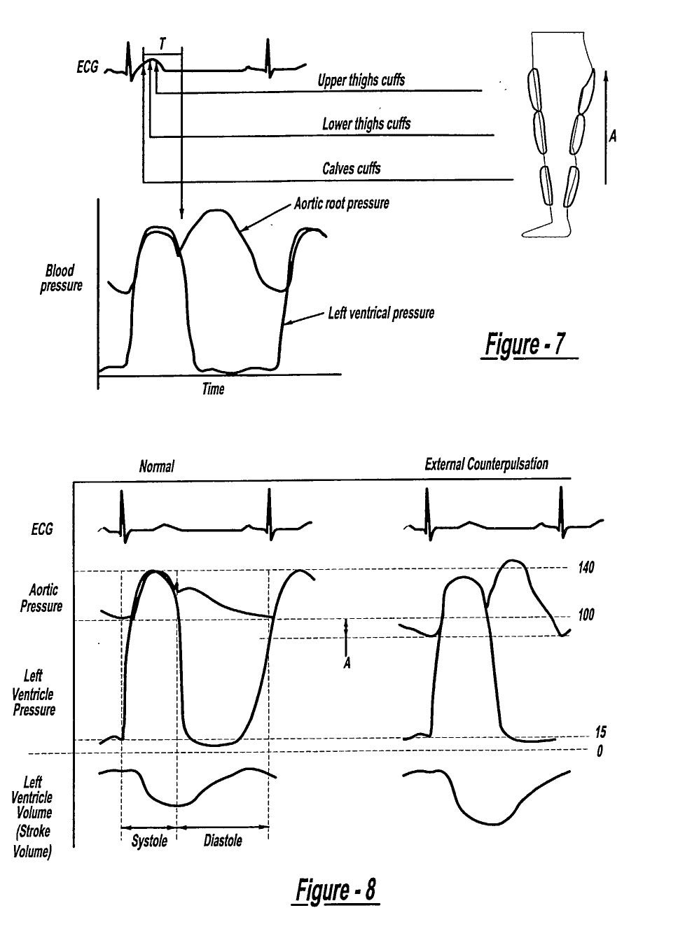

Manual adjustment of the ECP therapy comprises adjustment of the inflation time (varying C₁) and deflation time (C₂), so as to optimize efficacy. Deflation time is preferably adjusted before inflation time. The objective of adjusting deflation time from the R-wave or other triggering signals is to release all external pressure to achieve maximal decrease in end diastolic (or presystolic) aortic blood pressure. As demonstrated in FIG. 8, by lowering end diastolic pressure, the aortic valve opens earlier at a lower left ventricular pressure, the magnitude of which is indicated at A. The left ventricle, thus, spends less energy in isovolumetric contraction and reserves more energy for contraction. This increases stroke volume and cardiac output.

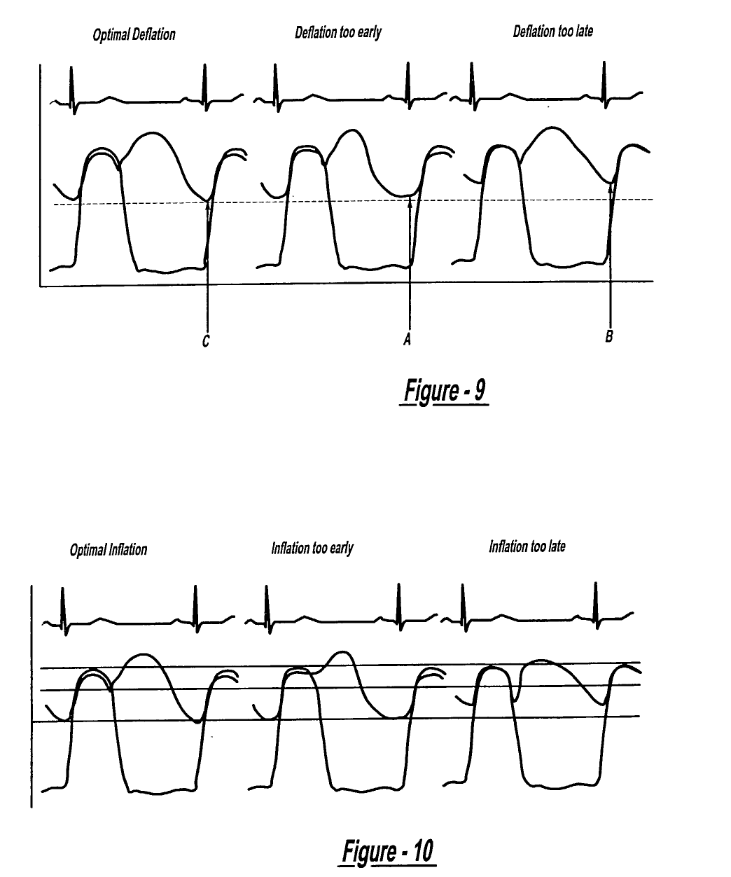

When deflation valves open too early, the end diastolic pressure is flat, as shown at A in the middle panel of FIG. 9. In this circumstance, blood from the upper portion of the body (i.e., head and neck) fills the peripheral vascular bed instead of the blood from the left ventricle, negating the “sucking effect” and reduction of systolic pressure by opening up the emptied vascular bed that has been previously compressed during diastole. In addition, opening the deflation valves too early reduces the area under the diastolic augmentation curve, thereby reducing coronary blood flow and energy supply to the myocardium. Further, if the deflation valves are opened too late, as shown at B in the right panel of FIG. 9, end diastolic pressure increases and the left ventricle spends more energy in isovolumetric contraction to generate more pressure before ejection begins. The left panel of FIG. 9 demonstrates timing the opening of the deflation valves to achieve maximum decrease in end diastolic (or pre-systolic) blood pressure, as indicated at C.

As mentioned before, the deflation time T₂ is initially set as (T_{R}−C₂), and C₂ is set as 160 ms; that is, the deflation time is set as 160 ms before the next R-wave. After this initial set-up, the operator is to adjust the deflation time manually to minimize the end diastole pressure. The first step is to adjust the deflation time earlier and observe the resulting changes in the patient pressure waveform to determine whether end diastolic pressure is lowered. If end diastolic pressure is lower, the deflation time is further adjusted to be earlier. If no change in end diastolic pressure is detected, then the deflation time is delayed to determine if there is any affect on end diastolic pressure. The procedures are repeated until the lowest end diastolic pressure is achieved with the latest possible deflation time. These steps ensure minimization of left ventricular isovolumetric contraction before ejection begins and maximization of diastolic augmentation by holding the diastolic compression as long as possible.

A diagrammatic representation of optimal inflation time is shown in the left panel of FIG. 10. If the inflation valves open too early (middle panel), retrograde pressure forces the aortic valves to close too early before the left ventricle finishes its ejection, thereby reducing cardiac output, and the applied external pressure becomes a load against which the heart must eject, thereby increasing cardiac workload. That is, the retrograde pressure is countered by left ventricle ejection when the myocardium is not in a relaxed state and resistance to coronary blood flow is still high. On the other hand, if the inflation valve opens too late (right panel), the myocardium has already begun to relax and blood pressure to the related coronary is not well-augmented to provide a high pressure to force open collateral channels and supplement coronary blood flow. Late opening also reduces diastolic augmentation and fails to increase energy supply to the myocardium. Optimal valve opening, and thus inflation, causes a retrograde pressure or flow wave to reach the root of the aorta just at the point of time when the left ventricle finishes its contraction and the aortic valve closes.

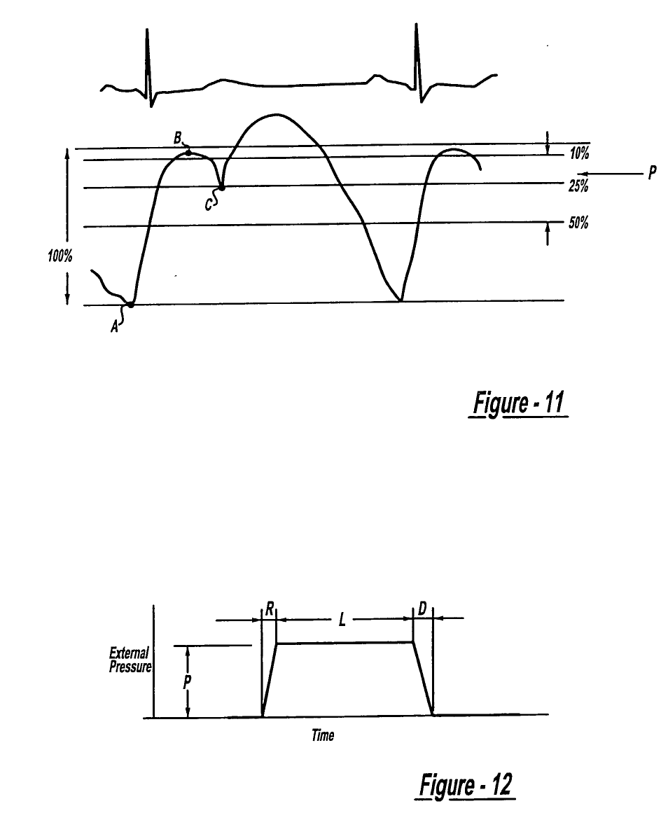

There are circumstances when the exact time at which aortic valve closes is difficult to detect, such as when noninvasive detection methods are used (e.g., a finger plethysmograph) and a dicrotic notch does not appear on the resulting waveform. The dicrotic notch may not be visible because it is composed of a high-frequency wave that is attenuated much faster when transmitting through the vascular bed. In this case, as exemplified in FIG. 11, the inflation time is adjusted by approximation, such that the diastolic waveform begins when the systolic pressure has dropped from about 10% to about 50% of the distance from the end diastolic pressure (A) to the peak systolic pressure (B). Thus, the pressure drop from peak systolic pressure (B) to the beginning of diastolic augmentation pressure (C) is from about 10% to about 50%, preferably from about 25% to about 50%, of the difference in pressure between the end diastolic pressure (A) and peak systolic pressure (B); or B−C=α(B−A), where α is from about 0.1 to about 0.5, preferably 0.25 to 0.5. This approximation, is derived from the observation that the counterpulsation pulse arrives at the root of the aorta, i.e., point C in FIG. 11, when the peak systolic pressure has dropped approximately 10 to 50 percent, as shown at P.

Concerning the externally applied pressure waveform, optimal operation of the ECP apparatus not only depends on proper inflation and deflation times, it also depends on the manner by which external pressure is applied, which is affected by the specific configuration of components of the apparatus. In particular, the time it takes for the applied pressure to rise to peak pressure (the rise time), the magnitude of the peak pressure, the length of time the pressure is applied (duration), and the speed with which the pressure is released (decay time), are considered.

Compression of the peripheral vascular bed produces a retrograde pulse that represents diastolic augmentation. The magnitude and velocity of propagation of the pressure wave up the aorta represents the potential and kinetic energy transmitted to the vasculature. The kinetic energy depends on the how fast the external pressure is applied, and the potential energy depends on the magnitude of the external pressure. Therefore, as shown in FIG. 12, the rate of rise time R, the magnitude of peak external pressure P, the time duration L of external pressure, and the time duration decay time D all contribute to the production of optimal pressure waveform.

The rise time R, or rate of applied pressure, depends on the rate of delivery of compressed fluid, which in turn depends on the magnitude of the compressed fluid in the reservoir (as high as possible but limited due to safety factors), the peak external pressure P to be applied to the body (200 to 320 millimeters Hg for patient safety and comfort), the dimension of the inflation valves and hoses (as large as practical, i.e., one-half to one inch in diameter), the volume of each pressure device to be inflated (dead space should be reduced as much as possible). An acceptable rise time R, for example, is 40 to 100 milliseconds to inflate from 0 to 320 millimeters Hg. The duration L of applied pressure is as long as possible, but is governed by inflation/deflation timing.

The peak external pressure P that can be applied to the body is the most important factor for effectiveness and safety considerations. In principle, one expects to apply a pressure slightly larger than the systolic pressure so as to collapse the conductive large arteries. However, that is typically not enough to compress the tissue surrounding the arteries, the microvessels that actually contain larger volume of blood. In addition, there is a loss of pressure from the skin layer through the muscle to the arteries. To achieve maximal diastolic augmentation, a peak tank pressure P of 250 to 350 millimeters Hg is preferred. For example, some patients with very calcified arteries, or patients with very stiff peripheral muscle, require a higher tank pressure up to 350 millimeters Hg. Beyond 350 millimeters Hg, the pressure is typically not safe to apply to a body because it may induce trauma to the skin and muscle.

The decay time D is defined as the time taken for the pressure devices to deflate from peak external pressure P to atmospheric pressure. The faster the decay time D, the more efficient the deflation to lower end diastolic pressure. In addition, when the heart rate of the patient under treatment is high (100-120 bpm), the time for inflation and deflation is limited to a very short period, and it becomes necessary to shorten the decay time D to less than 100 to 120 milliseconds. The implementation of fast decay is opening the deflation valves as large as possible, employing large diameter hoses, and, if necessary, using a vacuum source to suck the compressed air from the pressure devices, especially during the last portion of deflation when the pressure in the pressure devices is low. An acceptable rate of deflation, for example, is from about 40 to about 120 milliseconds to deflate from about 320 to about 0 millimeters Hg.

Also, preferably, the volume of peripheral muscle under compression is maximized by using a pressure device size with length covering all peripheral volume below the iliac crest. Compressing above the iliac crest is not recommended because it is difficult to transmit any externally applied pressure to the vasculature in the abdominal cavity and often results in patient movement rendering the effort useless. Nonetheless, where such pressure can be efficiently transmitted and patient movement retarded, further maximization can be achieved. In addition to producing better diastolic augmentation, the maximal peripheral volume under compression would also produce larger venous return and more emptied vasculature when deflated to receive left ventricle ejected blood volume to achieve a lower systolic pressure as well as end diastolic pressure.

Concerning the pressure gradient between sets of pressure devices, further optimization is achieved by applying peak external pressure in the distal pressure devices that is higher than the proximal pressure devices. Thus, in addition to inflating successive sets of pressure devices sequentially from distal to proximal regions of the body with time delay in an effort to “milk” the peripheral blood back up the vascular tree, the peak applied external pressure in the distal pressure devices is higher than the proximal pressure devices, as shown also in FIG. 4. The pressure gradient between successive sets of pressure devices prevents blood from leaking distally and thereby diminishing the volume of blood that can be squeezed back up the aorta. Creating too great a pressure drop between pressure devices, however, causes too low an external pressure to be applied in the proximal portion of the body, i.e., the upper thighs and buttocks that have larger blood volume. Thus, a pressure drop of from about 10 to about 30 millimeters Hg is desirable between successive set of pressure devices, depending the number of sets of pressure devices used. Further, a pressure of from about 200 to about 300 millimeters Hg applied to the distal pressure devices, and from about 160 to about 250 millimeters Hg applied to the proximal pressure devices, is preferred. Preferably external pressure is applied in an even manner to each of the pressure device so that the muscle, and specifically the vasculature, under each pressure devices will have a uniform applied pressure.

ECP apparatus useful in the methods of this invention are disclosed in the following patent documents, all of which are incorporated by reference herein: U.S. Pat. No. 3,303,841, Dennis, issued Feb. 14, 1967; U.S. Pat. No. 3,403,673, MacLeod, issued Oct. 1, 1968; U.S. Pat. No. 3,654,919, Birtwell, issued Apr. 11, 1972; U.S. Pat. No. 3,866,604, Curless et al., issued Feb. 18, 1975; U.S. Pat. No. 4,753,226, Zheng et al., issued Jun. 28, 1988; U.S. Pat. No. 5,554,103, Zheng et al., issued Sep. 10, 1996; U.S. Pat. No. 5,997,540, Zheng et al., issued Dec. 7, 1999; PCT Application WO 99/08644, Shabty et al., published Feb. 25, 1999; U.S. patent application Ser. No. 10/037,974, Hui, filed Nov. 9, 2001; U.S. patent application Ser. No. 10/037,874, Hui, filed Nov. 9, 2001; and U.S. patent application Ser. No. __/______, Hui, filed concurrently with the present application (attorney docket number 4857-000003). Preferred ECP apparatus useful herein include the MC-2 and TS-3 enhanced external counterpulsation therapy systems marketed by Vasomedical, Inc., Westbury, N.Y. U.S.A.

The present invention also provides ECP apparatus adapted for use in the treatment of heart failure. In such embodiments, the ECP apparatus comprises a controller or other device that facilitates use of the apparatus in the treatment of heart failure. Such facilitation may be through incorporation of monitors to monitor the safety or efficacy of the therapy; diagnostic devices; usage instructions, including instructions that are written or are part of the user interface; and control devices to, for example, minimize end diastolic pressure. The present invention also provides ECP systems comprising an ECP apparatus and usage instructions for a method of treating heart failure using ECP. Such usage instructions include one or more of instruction manuals, educational literature, and labeling that may be provided through modalities including written literature accompanying the apparatus or otherwise, the user interface in the apparatus (e.g., through prompts and instructions), customer service personnel (e.g., educational seminars, promotional interactions, and individual consultations), and remote computer interface (e.g., by modem or through the internet). A preferred system comprises:

(a) an ECP apparatus comprising (i) one or more pressure devices that are applied to an extremity of said subject; (ii) a device for inflating and deflating said pressure devices; and (iii) a controller that initiates inflation and deflation of said pressure devices in synchrony with the cardiac cycle of said subject; and

(b) instructions for the use of said apparatus for the treatment of heart failure.

The present invention provides methods of treating heart failure, including methods of facilitating treatment of heart failure by a medical service provider, comprising providing an ECP apparatus to the service provider and instructing the provider on the use of the ECP apparatus for the treatment of heart failure. As referred to herein, “providing” an ECP apparatus refers to any method of making an ECP apparatus available to a service provider (e.g., physician, nurse, medical technician or other individual administering ECP to a subject) for a purpose comprising the treatment of heart failure. Providing includes manufacturing an ECP apparatus for use in an ECP method and other activities including labeling and promotion. Instructing the provider includes methods for facilitating the use of the ECP apparatus in methods of this invention, including by providing one or more of instruction manuals, educational literature, and labeling that may be provided through modalities including written literature accompanying the apparatus or otherwise, the user interface in the apparatus (e.g., through prompts and instructions), customer service personnel (e.g., educational seminars, promotional interactions, and individual consultations), and remote computer interface (e.g., by modem or through the internet).

In a preferred embodiment, the methods of this invention also comprise monitoring the subject being treated with ECP for one or more indicia of safety or efficacy. Such indicia include those pertaining to blood oxygen level, respiration rate, heart rate, and diagnostic indicators of heart failure including evaluation of symptoms such as dyspnea, orthonea, paroxysmal nocturnal dyspnea, chronic fatigue, peripheral edema, ascites, tachycardia, “galloping” heart rhythm, rales, jugular venous distention, and oliguria; chest x-ray to assess the size of the heart; electrocardiogram, preferably with Doppler interrogation; radionuclide ventriculogram; coronary angiography; and magnetic resonance imaging.

A preferred monitoring step comprises monitoring of the subject's blood oxygen level. Such monitoring may be conducted through oximetry methods among those known in the art. Monitoring may be through devices incorporated into the ECP apparatus, or otherwise. In a preferred embodiment, oximeters function by measuring the oxygen saturation (the amount of oxygenated hemoglobin as a percentage of total hemoglobin) in arterial blood. In general, methods for measuring oxygen saturation utilize the relative difference between the light absorption (or attenuation) coefficient of oxygenated hemoglobin and that of reduced hemoglobin. The light absorption coefficient for oxygenated hemoglobin and reduced hemoglobin is dependent on the wavelength of the light. Oxygenated hemoglobin and reduced hemoglobin have different light absorption coefficients in the red and infrared regions. Thus, the two colors typically chosen to shine through the blood sample are red light and infrared light. In oximeters, light intensity is measured at various physiological states created by the pulsing of the vasculature as blood flows. As the heart beats, arterial blood is forced in the arteries and capillaries to produce a blood filled state. The blood then drains leaving a reference which consists of tissue, bone and some amount of venous blood. The collected transmitted light is subjected to photoelectric conversion and then mathematical conversion to eventually calculate the degree of oxygen saturation in the blood.

Oxygen saturation of blood may be determined “in vitro”, commonly in a container called a cuvette. Measurements are first made of the light transmitted through a cuvette filled with a saline solution. This provides a “bloodless” reference measurement for use in the oxygen saturation calculation. The cuvette is then filled with blood and a second set of measurements of transmitted light intensity is taken, to provide “blood-filled” measurements at two wavelengths. The foregoing measurements of light intensity are converted to absorption values and are then used with standard equations to solve for blood oxygen saturation.

In a preferred embodiment, non-invasive oximeters are used to measure oxygen saturation. Oximeters function by passing light of various colors or wavelengths through a sample. On the human body, typical measuring points are the tip of a finger or an ear lobe. The oximeter determines SpO₂ and pulse rate by passing two wavelengths of low intensity light, one red and one infrared, through body tissue to a photodetector. The sample absorbs the transmitted light to varying degrees relative to the particular constituents through which the light passes. A photosensitive device, such as a photo multiplier tube or photodiode, is used to detect the transmitted light. Alternatively, the photosensitive device can be designed to detect the light reflected from the sample. During measurement, the signal strength resulting from each light source depends on the color and thickness of the body tissue, the sensor placement, the intensity of the light sources, and the absorption of the arterial and venous blood (including the time varying effects of the pulse) in the body tissues. Either system provides a measure of the light the sample absorbs, i.e., the light the sample does not transmit or reflect. Using measurements of the transmitted light intensity, the absorption of light by the sample can be calculated. The oximeter processes these signals, separating the time invariant parameters (tissue thickness, skin color, light intensity, and venous blood) from the time variant parameters (arterial volume and SpO₂) to identify the pulse rate and calculate oxygen saturation.

A low oxygen saturation measurement is a warning of dangerous oxygen deprivation, or hypoxemia, a potential cause of injury or death. The specific minimum safe blood oxygen level is determined according to sound medical practice. In one method, the minimum blood oxygen circulating percentage is about 86%, preferably about 90%. In another method, the minimum level is a blood oxygen circulation percentage that is about 5 percentage points (on an absolute basis) less than the subject's blood oxygen level prior to initiation of an ECP treatment session. In one embodiment, therapy is terminated if the blood oxygen level drops below the minimum level. In another method, the oximeter or ECP apparatus provides the service provider with a visual or audible notification that the minimum level has been reached.

A preferred method comprises

(1) providing a plurality of inflatable devices adapted to be received about the lower extremities of said subject;

(2) interconnecting a source of compressed fluid with said inflatable devices;

(3) interconnecting a fluid distribution assembly with said inflatable devices and said source of compressed fluid;

(4) distributing compressed fluid from said source of compressed fluid to said inflatable devices;

(5) providing a controller in communication with said fluid distribution assembly;

(6) inflating and deflating said inflatable devices using said controller so as to minimize end diastolic pressure, whereby an increased cardiac output in said subject is achieved;

(7) measuring the oxygen level in the blood of said subject; and

(8) providing a warning to the operator or (preferably) terminating said inflating and deflating of the inflatable devices if said oxygen level drops below a safe level. In another embodiment, said inflating and deflating step is performed so as to maximize systolic unloading and diastolic augmentation. Preferably, the inflating and deflating step is performed so as to minimize end diastolic pressure and maximize systolic unloading and diastolic augmentation.

In a preferred embodiment, an ECP apparatus comprises a device for measuring blood oxygen level. Preferably in such a device, the ECP apparatus comprises a controller that monitors the blood oxygen level and determines if it falls below a safe level determined pursuant to sound medical practice, as discussed above. Such a level may be set by the service provider, or be automatically determined by the ECP apparatus. In one embodiment, the controller terminates therapy if the blood oxygen levels falls below the safe level. In another embodiment, the controller provides a visual or audible signal to the service provider.

In a preferred embodiment, the methods of this invention also comprise administering to the subject a drug for the treatment of heart failure. Such drugs useful herein include those that increase the force of contraction of the heart (by using inotropic agents), reducing fluid accumulation in the lungs and elsewhere (by using diuretics), and reducing the work load of the heart (by using agents that decrease systemic vascular resistance). Such drugs useful herein include digoxin, digitoxin, quabain, and other cardiac glycosides; furosemide, bumetamide, torsemide, and other loop diuretics; thiazide diuretics; potassium-sparing diuretics; losartin, captopril, enalapril, enalaprilat, quinapril, lisinopril, ramipril, and other angiotensin converting enzyme inhibitors; losartan and other angiotensin receptor antagonists; aminorone, milrinone, vesnarinone and other phosphodiesterase inhibitors; sodium nitroprusside, glutathione, nitroglycerin, isosorbide dinitrate, and other nitrovasodilators; hydralazine, nicorandil and other direct vasodilators; prazosin, phentolamine, labetalol, carvedilol, bucindolol, and other andrenergic receptor antagonists; verapamil, diltiazem and other benzothiopenes, nifedipine, amlodipine and other dihydropyridines, and other calcium channel antagonists; dopamine, dobutamine and other sympathomimetics; lovastatin, simvastatin, pravastatin, flavastatin and other HMB Co A reductase inhibitors; plasminogen, α₂-antiplasmin, streptokinase, tissue plasminogen activator, urokinase, and other antithrombolytics; and combinations thereof.

The examples and other embodiments described herein are exemplary and not intended to be limiting in describing the full scope of compositions and methods of this invention. Equivalent changes, modifications and variations of specific embodiments, materials, compositions and methods may be made within the scope of the present invention, with substantially similar results.

What is claimed is:

1. A method of treating heart failure in a human or animal subject, comprising administering a plurality of external counterpulsation therapy sessions to said subject during a treatment period of from about 10 to about 80 days.

2. A method according to claim 1, comprising from 1 to 3 sessions of external counterpulsation therapy during each day of at least 4 days of every 7-day period during a treatment period of from about 20 to about 60 days.

3. A method according to claim 2, wherein said counterpulsation therapy is administered for from about 20 to about 90 minutes per session.

4. A method according to claim 3, wherein said therapy is administered for from about 45 minutes to about 60 minutes per session.

5. A method according to claim 1, wherein said subject has systolic heart failure.

6. A method according to claim 1, wherein said subject has diastolic heart failure.

7. A method according to claim 1, wherein said administering of external counterpulsation therapy comprises
(a) providing a plurality of pressure devices adapted to be received about the lower extremities of said subject;
(b) interconnecting a source of compressed fluid with said pressure devices;
(c) interconnecting a fluid distribution assembly with said pressure devices and said source of compressed fluid;
(d) distributing compressed fluid from said source of compressed fluid to said pressure devices;
(e) providing a controller in communication with said fluid distribution assembly; and
(f) inflating and deflating said pressure devices using said controller so as to minimize end diastolic pressure, whereby an increased cardiac output in said subject is achieved.

8. A method according to claim 7, wherein said inflating and deflating step is performed so as to maximize systolic unloading and diastolic augmentation.

9. A method according to claim 7, additionally comprising measuring said subject's blood oxygen level.

10. A method according to claim 1, additionally comprising treating said subject with a drug selected from the group consisting of cardiac glycosides; loop diuretics; thiazide diuretics; potassium-sparing diuretics; angiotensin converting enzyme inhibitors; angiotensin receptor antagonists; phosphodiesterase inhibitors; nitrovasodilators; direct vasodilators; andrenergic receptor antagonists, calcium channel antagonists; sympathomimetics; HMG Co A reductase inhibitors; antithrombolytic agents, and combinations thereof.

11. A method according to claim 10, wherein said drug is selected from the group consisting of digoxin, digitoxin, quabain, furosemide, bumetamide, torsemide, losartin, captopril, enalapril, enalaprilat, quinapril, lisinopril, ramipril, losartan aminorone, milrinone, vesnarinone sodium nitroprusside, glutathione, nitroglycerin, isosorbide dinitrate, hydralazine, nicorandil prazosin, phentolamine, labetalol, carvedilol, bucindolol, verapamil, diltiazem, nifedipine, amlodipine dopamine, dobutamine, lovastatin, simvastatin, pravastatin, flavastatin, and combinations thereof.

12. A method according to claim 1, additionally comprising the step of performing a diagnostic on said subject, prior to said administering of external counterpulsation, to confirm the existence of said heart failure.

13. A method according to claim 12, wherein said diagnostic comprises clinical evaluation; chest radiography; computerized tomography; coronary magnetic resonance imaging; radionuclide ventriculogram, coronary angiography, electrocardiography; blood pressure monitoring; blood testing; pulse oximetry; echocardiography; hemodynamic evaluation; exercise stress testing; radionuclide stress perfusion testing; endomyocardial biopsy; and combinations thereof.

14. A method according to claim 13, additionally comprising the step of performing a diagnostic on said subject after said administering of external counterpulsation.

15. A method of treating heart failure in a human or other animal subject, comprising the steps of:
(a) performing a diagnostic on said subject to confirm the existence of heart failure; and if said heart failure is confirmed; and
(b) administering to said subject a plurality of external counterpulsation therapy sessions during a treatment period of from about 20 to about 60 days.

16. A method according to claim 15, wherein said diagnostic comprises a method selected from the group consisting of clinical evaluation; chest radiography; computerized tomography; coronary magnetic resonance imaging; radionuclide ventriculogram, coronary angiography, electrocardiography; blood pressure monitoring; blood testing; pulse oximetry; echocardiography; hemodynamic evaluation; exercise stress testing; radionuclide stress perfusion testing; endomyocardial biopsy; and combinations thereof.

17. A method of treating heart failure in a human or other animal subject, comprising the steps of:
(a) administering to said subject an external counterpulsation therapy session lasting from about 20 minutes to about 90 minutes and repeating said therapy session for from 1 to 3 times per day for at least about 70% of the days during a treatment period of from about 20 to about 80 days; and
(b) monitoring said subject to assess the safety and or efficacy of said therapy session.

18. A method according to claim 17, wherein said administering of external counterpulsation therapy comprises:
(1) providing a plurality of inflatable devices adapted to be received about the lower extremities of said subject;
(2) interconnecting a source of compressed fluid with said inflatable devices;
(3) interconnecting a fluid distribution assembly with said inflatable devices and said source of compressed fluid;
(4) distributing compressed fluid from said source of compressed fluid to said inflatable devices;
(5) providing a controller in communication with said fluid distribution assembly;
(6) inflating and deflating said inflatable devices using said controller so as to minimize end diastolic pressure, whereby an increased cardiac output in said subject is achieved;
(7) measuring the oxygen level in the blood of said subject; and
(8) providing a warning or terminating said inflating and deflating if said oxygen level drops below a safe level.

19. A method according to claim 18, wherein said measuring and said terminating steps are performed by said controller.

20. A method according to claim 18, wherein said inflating and deflating step is performed so that the pulse generated by application of an external pressure by said proximal inflatable device in compressing the vascular bed travels up the arterial tree and reaches the root of the aorta at approximately the same time as the closure of the aortic valve in said subject.

21. A system for performing external counterpulsation for the treatment of heart failure in a human or other animal subject, comprising:
(a) an ECP apparatus comprising (i) one or more pressure devices that are applied to an extremity of said subject; (ii) a device for inflating and deflating said pressure devices; and (iii) a controller that initiates inflation and deflation of said pressure devices in synchrony with the cardiac cycle of said subject; and
(b) instructions for the use of said apparatus for the treatment of heart failure.

22. A system according to claim 21, wherein said treatment comprises inflating and deflating said pressure devices so as to minimize end diastolic pressure and to maximize systolic unloading and maximize diastolic augmentation.

23. A system according to claim 22, wherein said treatment additionally comprises monitoring the blood oxygen level of said subject.

24. A system according to claim 23, wherein said ECP apparatus additionally comprises a device for measuring the blood oxygen level in said subject.
